SEQUENCE LISTING (6 sequences)

SEQ ID NO: 1 - DNA, 44 bp, Artificial
  Synthesized primer FIP
tacacctttgttcgagtcatgatgaaaggtttgagatattccca

SEQ ID NO: 2 - DNA, 41 bp, Artificial
  Synthesized primer BIP
ctcatgctggagcaaaaagcttcatttgctgagctttgggt

SEQ ID NO: 3 - DNA, 20 bp, Artificial
  Synthesized primer F3
gcaattgagctcagtgtcat

SEQ ID NO: 4 - DNA, 25 bp, Artificial
  Synthesized primer B3
tctttccctttatcattaatgtagg

SEQ ID NO: 5 - DNA, 18 bp, Artificial
  Synthesized primer LF
tgggccatgaacttgtct

SEQ ID NO: 6 - DNA, 23 bp, Artificial
  Synthesized primer LB
ggctagttaaaaaaggaaattca

(19) United States
(12) Patent Application Publication
(10) Pub. No.: US 2011/0312036 A1
(43) Pub. Date: Dec. 22, 2011

(54) MICROCHIP FOR ISOTHERMAL NUCLEIC-ACID AMPLIFICATION REACTION, METHOD FOR PRODUCING THE SAME, AND ISOTHERMAL NUCLEIC-ACID AMPLIFICATION METHOD

(75) Inventors: Kensuke Kojima, Kanagawa (JP); Tasuku Yotoriyama, Tokyo (JP); Tomohiko Nakamura, Tokyo (JP); Hidetoshi Watanabe, Chiba (JP); Yuji Segawa, Tokyo (JP)

(73) Assignee: SONY CORPORATION, Tokyo (JP)

(21) Appl. No.: 13/158,935

(22) Filed: Jun. 13, 2011

(30) Foreign Application Priority Data
Jun. 22, 2010 (JP) 2010-141318
Sep. 1, 2010 (JP) 2010-195780

(51) Int. Cl. C12P 19/34; B05D 3/00; C12M 1/00

(52) U.S. Cl. 435/91.2; 435/289.1; 427/238

(57) ABSTRACT

A microchip for an isothermal nucleic-acid amplification reaction in which at least one of substances necessary for an isothermal amplification reaction of a nucleic acid is present in a reaction region functioning as a reaction field of the reaction, the at least one of the substances being covered with a thin film that melts at a temperature higher than room temperature and lower than a reaction temperature of the reaction.

### CROSS REFERENCES TO RELATED APPLICATIONS

The present application claims priority to Japanese Priority Patent Application JP 2010-141318 filed in the Japan Patent Office on Jun. 22, 2010 and Japanese Priority Patent Application JP 2010-195780 filed in the Japan Patent Office on Sep. 1, 2010, the entire contents of which are hereby incorporated by reference.

### BACKGROUND

The present application relates to a microchip for an isothermal nucleic-acid amplification reaction, a method for producing the microchip, and an isothermal nucleic-acid amplification method. More specifically, the present application relates to a microchip for an isothermal nucleic-acid amplification reaction, the microchip being configured to accurately controlling the reaction time of a nucleic acid amplification reaction etc.

Hitherto, a polymerase chain reaction (PCR) method has been used as a nucleic acid amplification method. In the PCR method, a nucleic acid strand serving as a template is amplified by repeating a temperature cycle including three steps of (1) thermal denaturation, (2) annealing, and (3) elongation reaction.

The thermal denaturation in step (1) is a step of dissociating the template nucleic acid strand from a double strand to a single strand. The reaction temperature during the thermal denaturation is usually about 94° C. The annealing in step (2) is a step of bonding oligonucleotide primers to the template nucleic acid strands each of which has been dissociated into a single strand. The reaction temperature during the annealing is usually about 50° C. to 60° C. The elongation reaction in step (3) is a step of synthesizing, with a DNA polymerase, DNA molecules that are complementary to the corresponding single strand, the synthesis being started from the portion to which the oligonucleotide primer has been bonded. The reaction temperature during the elongation reaction is usually about 72° C.

Recently, a method called isothermal amplification method, which is a simpler method that does not need repetition of a temperature cycle, has been used as the nucleic acid amplification method. For example, in a loop-mediated isothermal amplification (LAMP) method, a reaction is carried out by mixing a template nucleic acid strand with reagents such as oligonucleotide primers, a strand displacement DNA polymerase, and a nucleic acid monomer, and maintaining the resulting mixture at a constant temperature (about 65° C.). Thus, according to this LAMP method, a nucleic acid can be amplified in one step.

In association with the present application, recently, nucleic acid amplification devices (e.g., real-time PCR devices) have been developed in which a nucleic acid amplification reaction is conducted in a well of a microchip, and amplified nucleic acid strands are optically detected or quantified.

Japanese Unexamined Patent Application Publication No. 2007-43998 discloses a micro-fluid chip in which an oligonucleotide primer, a substrate, an enzyme, and other reagents, all of which are necessary for a nucleic acid amplification reaction, are placed in a solid state in a channel. In this micro-fluid chip, when other reagents necessary for the reaction are sent to the channel in a liquid state, the reagents in the liquid state and the reagents in the solid state contact each other and the reagents in the solid state are dissolved, thereby staring the reaction.

### SUMMARY

In the PCR method, a method called “hot-start method” is employed in order to strictly control the reaction time. The hot-start method is a method in which a nonspecific amplification reaction due to misannealing of an oligonucleotide primer is prevented, and a desired amplified product is obtained. The hot-start method is achieved by heating a mixed liquid containing a target nucleic acid strand and reagents other than a DNA polymerase to a denaturation temperature of an oligonucleotide primer, adding the enzyme at the denaturation temperature, and then performing a temperature cycle.

In contrast, in the isothermal amplification method, since a reaction is conducted in one step at a constant temperature, the hot-start method is not used. Consequently, the reaction gradually proceeds at the time when reagents and a template nucleic acid strand are mixed, and thus it is difficult to strictly control the reaction time.

Hitherto, in a microchip-type nucleic acid amplification device, a method in which reagents and a template nucleic acid strand are mixed in advance, and the resulting mixed liquid is then introduced into a well of a microchip to conduct a reaction has been employed. Accordingly, the reaction may proceed in the mixed liquid during the preparation of the mixed liquid or during the introduction of the mixed liquid into the well. Thus, it is difficult to strictly control the reaction time, resulting in a problem in terms of quantitativity of amplified nucleic acid strands.

It is desirable to provide a technology for accurately control the reaction time in an isothermal amplification method.

According to an embodiment, there is provided a microchip for an isothermal nucleic-acid amplification reaction in which at least one of substances necessary for an isothermal amplification reaction of a nucleic acid is present in a reaction region functioning as a reaction field of the reaction, the at least one of the substances being covered with a thin film that melts at a temperature higher than room temperature and lower than a reaction temperature of the reaction.

In the microchip for an isothermal nucleic-acid amplification reaction, at least one of the remaining substances necessary for the reaction is preferably fixed on the thin film covering the at least one of the substances necessary for the reaction.

In the microchip for an isothermal nucleic-acid amplification reaction, the at least one of the substances present in the reaction region may be at least one selected from an oligonucleotide primer, an enzyme, and a nucleic acid monomer.

In this microchip for an isothermal nucleic-acid amplification reaction, the reaction can be started at any timing by melting the thin film by heating, the thin film covering the at least one of the substances placed in advance in the reaction region, after a sample solution containing the remaining substances and a target nucleic acid strand is supplied to the reaction region.

In the microchip for an isothermal nucleic-acid amplification reaction, an oligonucleotide primer is preferably fixed on the thin film covering an enzyme.

In the microchip for an isothermal nucleic-acid amplification reaction, the thin film is preferably formed by evaporation of stearic acid or a paraffin wax.

The microchip for an isothermal nucleic-acid amplification reaction preferably includes an inlet into which a liquid is introduced from the outside, a plurality of the reaction regions, and a channel configured to supply the liquid introduced from the inlet to the reaction regions.

According to another embodiment, there is provided a method for producing a microchip for an isothermal nucleic-acid amplification reaction, the method including covering at least one of substances necessary for an isothermal amplification reaction of a nucleic acid, the at least one of the substances being placed in a reaction region functioning as a reaction field of the reaction, with a thin film that melts at a temperature higher than room temperature and lower than a reaction temperature of the reaction.

According to another embodiment, there is provided an isothermal nucleic-acid amplification method including, into a reaction region where at least one of substances necessary for an isothermal amplification reaction of a nucleic acid is present, the at least one of the substances being covered with a thin film that melts at a temperature higher than room temperature and lower than a reaction temperature of the reaction, introducing the remaining substances necessary for the reaction; and increasing the temperature to the reaction temperature.

In the embodiments, “isothermal nucleic-acid amplification reaction” includes various amplification reactions that involve no temperature cycle. Examples of the isothermal amplification reactions include a loop-mediated isothermal amplification (LAMP) method, a SMart Amplification Process (SMAP), a nucleic acid sequence-based amplification (NASBA) method, an isothermal and chimeric primer-initiated amplification of nucleic acids (ICAN) method (registered trademark), a transcription-reverse transcription concerted (TRC) method, a strand displacement amplification (SDA) method, a transcription-mediated amplification (TMA) method, and a rolling circle amplification (RCA) method. In addition, “nucleic acid amplification reaction” widely includes nucleic acid amplification reactions at a constant temperature for the purpose of amplification of nucleic acids. These nucleic acid amplification reactions also include a reaction which involves quantitative determination of amplified nucleic acid strands in addition to amplification of a nucleic acid strand, for example, a real-time (RT)-LAMP method.

“Substances necessary for a reaction” refer to substances necessary for obtaining an amplified nucleic acid strand in an isothermal nucleic-acid amplification reaction. Specific examples of the substances include oligonucleotide primers each having a base sequence complementary to a target nucleic acid strand, a nucleic acid monomer (deoxynucleotide-triphosphate (dNTP)), enzymes, and solutes of a reaction buffer.

According to an embodiment, there is provided a technology for accurately control the reaction time in an isothermal amplification method.

Additional features and advantages are described herein, and will be apparent from the following Detailed Description and the figures.

### BRIEF DESCRIPTION OF THE FIGURES

FIG. 1 is a schematic top view of a microchip for a nucleic acid amplification reaction according to an embodiment;

FIG. 2 is a schematic cross-sectional view of the microchip (i.e., cross-sectional view taken along line II-II in FIG. 1);

FIGS. 3A to 3C are schematic views illustrating a substance necessary for a reaction, the substance being present in a well of a microchip;

FIG. 4 is a flowchart illustrating a method for producing a microchip for a nucleic acid amplification reaction according to an embodiment;

FIGS. 5A to 5C are schematic views illustrating substances necessary for a reaction, the substances being present in a well of a microchip according to a modification;

FIG. 6 is a flowchart illustrating a method for producing a microchip for a nucleic acid amplification reaction according to a modification;

FIG. 7 is a chart showing the results of a LAMP reaction in a reaction system in which a primer is fixed by covering with stearic acid or a paraffin wax (Example 2); and

FIG. 8 is a chart showing the results of a LAMP reaction in a reaction system in which an enzyme is fixed by covering with stearic acid or a paraffin wax (Example 2).

### DETAILED DESCRIPTION

Embodiments of the present application will be described below in detail with reference to the drawings.

Embodiments for carrying out the present application will now be described with reference to the drawings. The embodiments described below illustrate only examples of typical embodiments, and the scope of the present application is not narrowly interpreted by the embodiments.

1. Microchip for Isothermal Nucleic-Acid Amplification Reaction and Isothermal Nucleic-Acid Amplification Method

[Microchip for Isothermal Nucleic-Acid Amplification Reaction]

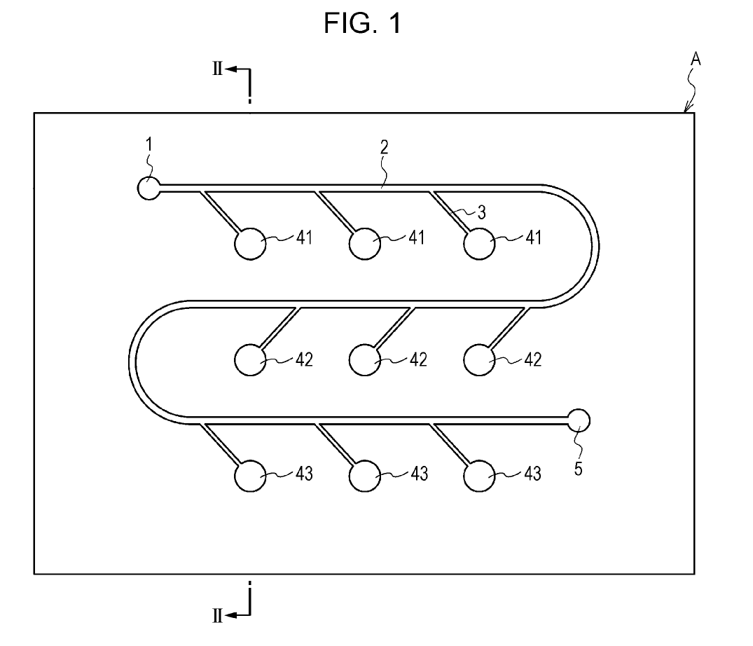

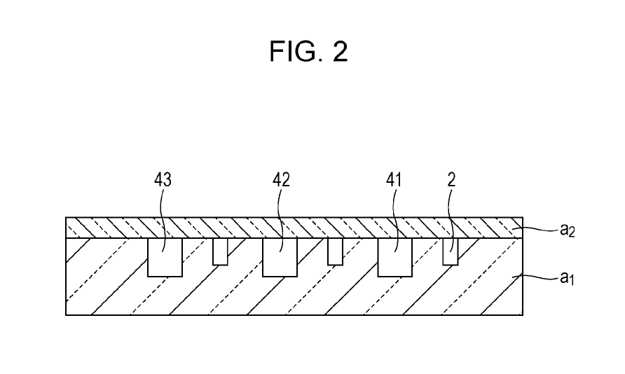

FIG. 1 is a schematic top view of a microchip for an isothermal nucleic-acid amplification reaction (hereinafter, also simply referred to as “microchip”) according to an embodiment, and FIG. 2 is a schematic cross-sectional view of the microchip. FIG. 2 corresponds to a cross section taken along line II-II in FIG. 1.

A microchip A includes an inlet 1 into which a liquid (sample solution) is introduced from the outside, a plurality of wells (reaction regions) functioning as reaction fields of a nucleic acid amplification reaction, a main channel 2 that communicates with the inlet 1 at an end thereof, and branch channels 3 branching from the main channel 2. Another end of the main channel 2 functions as an outlet 5 that discharges the sample solution to the outside. The branch channels 3 branch from the main channel 2 at positions between a portion communicating with the inlet 1 and a portion communicating with the outlet 5 of the main channel 2, and are each connected to the corresponding well. The sample solution can contain DNA, genome RNA, mRNA, or the like that serves as a template nucleic acid strand in the nucleic acid amplification reaction.

In this embodiment, a total of nine wells are arranged at uniform intervals in three rows and three columns in the microchip A. These nine wells are divided into three groups. The three wells in the upper row in FIG. 1 are indicated by reference numeral 41, the three wells in the middle row in FIG. 1 are indicated by reference numeral 42, and the three wells in the lower row in FIG. 1 are indicated by reference numeral 43. The sample solution introduced from the inlet 1 is sent through the main channel 2 toward the outlet 5, and is sequentially supplied inside the branch channels 3 and the wells from upstream in a solution-sending direction. In the microchip A, the outlet 5 may not be provided. Specifically, the microchip A may be configured so that the sample solution introduced from the inlet 1 is not discharged to the outside.

The microchip A is produced by bonding a substrate layer a₂ to a substrate layer a₁ in which the inlet 1, the main channel 2, the branch channels 3, the wells 41, 42, and 43, and the outlet 5 are formed. The materials of the substrate layers a₁ and a₂ may be glass or a plastic such as polypropylene, polycarbonate, a cycloolefin polymer, or polydimethylsiloxane (PDMS). When detection or quantitative determination of nucleic acid strands amplified in the wells 41, 42, and 43 is performed by an optical method, the materials of the substrate layers a₁ and a₂ are preferably selected from materials having optical transparency and low optical errors because of a low autofluorescence and small wavelength dispersion.

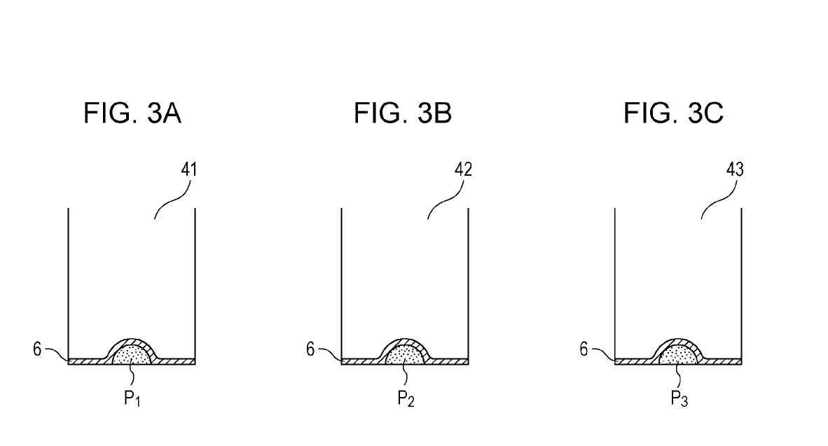

At least one of substances necessary for a reaction is present in the wells 41, 42, and 43, the at least one of the substances being covered with a thin film having a heat-melting property. FIGS. 3A to 3C show a substance placed in the wells. FIG. 3A shows a substance placed in the well 41, FIG. 3B shows a substance placed in the well 42, and FIG. 3C shows a substance placed in the well 43.

The substance present in the well is a substance necessary for obtaining amplified nucleic acid strands in an isothermal nucleic-acid amplification reaction. Specifically, the substance is selected from an oligonucleotide primer having a base sequences complementary to a target nucleic acid strand, a nucleic acid monomer (dNTP), an enzyme, a solutes of a reaction buffer, and the like. One or more of these substances may be present in each of the wells. Also, a reagent necessary for detecting and quantitatively determining amplified nucleic acid strands, for example, a fluorescence reagent (fluorescent dye) or a phosphorescent reagent (phosphorescent dye), may be optionally placed in the wells, though such a reagent is not necessarily used for obtaining the amplified nucleic acid strands.

FIGS. 3A to 3C show a case where oligonucleotide primers (hereinafter, also simply referred to as “primers”) P₁, P₂, and P₃ are placed in the wells 41, 42, and 43, respectively.

The primers P₁, P₂, and P₃ may be primers having the same base sequence. However, when a plurality of target nucleic acid strands are amplified in the microchip A, primers having different base sequences are used as the primers P₁, P₂, and P₃. For example, when a genotype is determined using the microchip A, primers having base sequences that are different depending on base sequences of respective genotypes are respectively placed in the wells 41, 42, and 43. Similarly, when a contagium is determined using the microchip A, primers having base sequences that are different depending on gene sequences of respective viruses or microorganisms are respectively placed in the wells.

The primers P₁, P₂, and P₃ in the wells are each covered with a thin film 6 that melts with heat at a temperature higher than room temperature and lower than the reaction temperature of the isothermal nucleic-acid amplification reaction.

The thin film 6 is formed of a material that loses fluidity and enters a solid state at temperatures (including room temperature) lower than the melting temperature, and that is melted or fluidized and enters a liquid state or a gel state at temperatures equal to or higher than the melting temperature. At temperatures lower than the melting temperature, each of the primers P₁, P₂, and P₃ covered with the thin film 6 is present inside the corresponding solid thin film 6 so as to be isolated from the outside. At temperatures equal to or higher than the melting temperature, each of the primers P₁, P₂, and P₃ is released from the corresponding thin film 6 that is fluidized and collapsed, and can be in contact with the outside.

The reaction temperature of the isothermal nucleic-acid amplification reaction is usually in the range of 50° C. to 75° C. The melting temperature or the fluidizing temperature of the thin film 6 is determined so as to be higher than about 25° C., which is room temperature, and lower than 50° C. to 75° C., which is the reaction temperature. By designing the melting temperature or the like of the thin film 6 within this range, the primers P₁, P₂, and P₃ can be reliably isolated from the outside at temperatures lower than the melting temperature, and can be rapidly released at temperatures equal to or higher than the melting temperature.

In order to reliably isolate the primers P₁, P₂, and P₃ from the outside at temperatures lower than the melting temperature, the thin film 6 preferably has water repellency. By imparting water repellency to the thin film 6, it is possible to prevent the thin film 6 from being dissolved by the sample solution introduced in the wells at temperatures lower than the melting temperature.

The material of the thin film 6 is not particularly limited as long as the material has the above melting temperature or the like and preferably has water repellency. Examples of the material of the thin film 6 include fatty acids such as stearic acid, palmitic acid, and myristic acid, behenyl alcohol, agarose, paraffin wax, microcrystalline wax, and gelatin.

As for the melting point of these materials, stearic acid has a melting point of 69° C., palmitic acid has a melting point of 63° C., myristic acid has a melting point of 54° C., behenyl alcohol has a melting point of 65° C. to 73° C., agarose has a melting point of 65° C., paraffin wax has a melting point of 40° C. to 70° C., microcrystalline wax has a melting point of 60° C. to 90° C., and gelatin has a melting point of 40° C. to 50° C.

Examples of the material of the thin film 6 also include natural materials such as carnauba wax obtained from a secretion from leaves of a palmae plant and containing a large amount (30% to 35%) of a hydroxy acid ester; candelilla wax obtained from a plant grown in a dry region in the southern part of the North Africa and having a high proportion (40% to 50%) of hydrocarbons; vegetable wax obtained from nuts of Japanese wax tree and containing glyceride as a main component, and other vegetable hydrogenated fat and oil. As for the melting point of these materials, carnauba wax has a melting point of 80° C. to 86° C., candelilla wax has a melting point of 66° C. to 71° C., vegetable wax has a melting point of 50° C. to 56° C., and vegetable hydrogenated fat and oil usually have a melting point of 48° C. to 70° C.

Furthermore, as the material of the thin film 6, the following compounds also have the above-mentioned melting temperature or the like. Examples thereof further include Nb(NtC₅H₁₁)[N(CH₃)₂]₃ (melting point: 47° C.), tris(ethylcyclopentadienyl)praseodymium (Pr(EtCp)₃) (melting point: 70° C. to 73° C.), which is a cyclopentadienyl (Cp) complex; Ta(NtC₅H₁₁N(CH₃)₂]₃ (melting point: 36° C.), which is used as a material for forming a TaN barrier film; triethylene glycol-bis[3-(3-tert-butyl-5-methyl-4-hydroxyphenyl)propionate (melting point: 76° C. to 79° C.), which is an antioxidant for styrene resins; 2,2-thio-diethylenebis[3-(3,5-di-tert-butyl-4-hydroxyphenyl)propionate (melting point: 63° C. or higher) and octadecyl-3-(3,5-di-tert-butyl-4-hydroxyphenyl)propionate (melting point: 50° C. to 53° C.), which are used as an antioxidant; baking soda (melting point: 70° C.); and 1-ethyl-3-methylimidazolium bifluoride (melting point: 51° C.), which is an ionic liquid.

As for the material of the thin film 6, it is preferable to select a material that does not inhibit a nucleic acid amplification reaction when the material is melted and mixed in a sample solution. As described in Examples below, for example, stearic acid and paraffin wax do not have a reaction inhibiting property. Alternatively, it is desirable to reduce the thickness of the thin film 6 to the extent that reaction inhibition is negligible when the thin film 6 is melted and mixed in a sample solution. The thickness of the thin film 6 is preferably controlled to be, for example, 1,000 nm (volume: 1 nL) or less. In this case, when the thin film 6 melts, the concentration of the material in the sample solution can be reduced to about 0.1%, and thus the reaction inhibition can be prevented. The thickness of the thin film 6 can be controlled to be any desired value in a range of about 1,000 nm to 10 nm by forming the thin film 6 by evaporation of the above material.

[Isothermal Nucleic-Acid Amplification Method]

Next, an isothermal nucleic-acid amplification method using the microchip A will be described.

First, a sample solution containing a target nucleic acid strand and substances (such as an enzyme, dNTP, and a buffer solute) necessary for a reaction, the substances being other than the primers which are placed in the wells in advance and then covered with the thin film 6, is supplied from the inlet 1 to each of the wells. At this time, the sample solution is maintained at room temperature, and thus the primers each covered with the thin film 6 are isolated from the sample solution. Accordingly, a reaction caused by mixing of the primers and the sample solution does not proceed.

After the sample solution is supplied to each of the wells, the temperature of the sample solution is increased to a reaction temperature. As a result, the thin film 6 is fluidized and collapsed so that each of the primers is released and mixed with the sample solution. Thus, a reaction is started.

As described above, in the microchip A, the reaction is started by supplying the sample solution to each of the wells and then increasing the temperature of the sample solution to the reaction temperature. Thus, the reaction time can be strictly controlled in the microchip A.

In this embodiment, a case where, as a substance necessary for a reaction, a primer covered with a thin film 6 is placed in a well in advance has been described as an example. Alternatively, the substance placed in the well may be an enzyme, dNTP, or a buffer solute. Alternatively, two or more substances, e.g., a primer and an enzyme; a primer and dNTP; or a primer, an enzyme, and dNTP may be placed in combination in the well.

In the microchip according to an embodiment, a substance necessary for a reaction is placed in advance in wells, the substance being covered with a thin film having a heat melting property, a sample solution containing remaining substances and a target nucleic acid strand is supplied to the wells, and the temperature is then increased to the reaction temperature. Thus, the reaction can be started at any desired timing. According to this microchip, the reaction time can be strictly controlled and amplified nucleic acid strands can be quantitatively determined with high accuracy.

In the microchip according to an embodiment, the number of wells and the arrangement positions of the wells are not limited, and the shape of the wells is also not limited to the columnar shape shown in the figures. The structures of the channels for supplying the sample solution introduced into the inlet 1 to each of the wells are also not limited to the structures of the main channel 2 and the branch channels 3 shown in FIG. 1. Furthermore, in this embodiment, a case where the inlet 1 and other components are formed in the substrate layer a₁ has been described. Alternatively, some of these components may be formed in the substrate layer a₁, and the other components may be formed in the substrate layer a₂. The number of substrate layers constituting the microchip may be 2 or more.

2. Method for Producing Microchip for Nucleic Acid Amplification Reaction

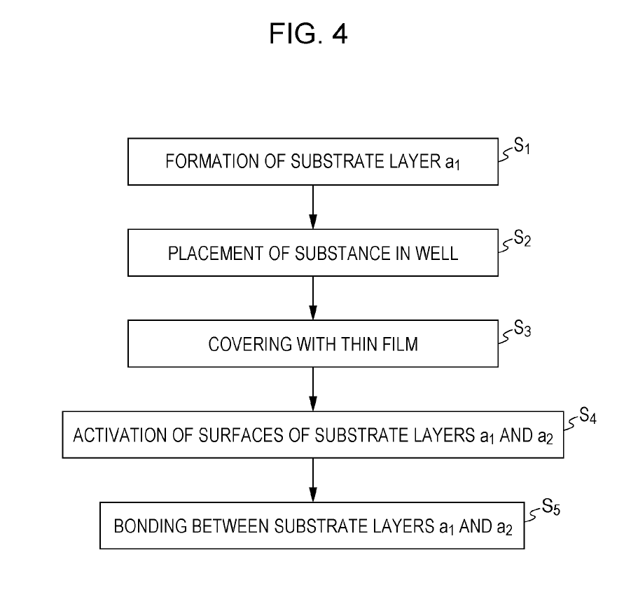

A method for producing the microchip according to an embodiment will now be described with reference to the flowchart shown in FIG. 4. A description will be made by taking the above-described microchip A as an example.

(2-1) Formation of Substrate Layer a₁

In FIG. 4, symbol S₁ shows a step of forming a substrate layer a₁. In this step, an inlet 1, a main channel 2, branch channels 3, wells 41, 42, and 43, and an outlet 5 are formed in the substrate layer a₁. The inlet 1 and other components can be formed in the substrate layer a₁ by, for example, wet etching or dry etching of a glass substrate layer or nanoimprinting, injection molding, or machining of a plastic substrate layer.

(2-2) Placement of Substance in Well

Symbol S₂ shows a step of placing a substance necessary for a reaction in the wells. In this step, solutions of primers P₁, P₂, and P₃ are dropped in the wells 41, 42, and 43, respectively, and dried, thus placing the primers in the wells. As described above, the substance placed in the wells is not limited to an oligonucleotide primer. Alternatively, for example, dNTP, an enzyme, or a buffer solute may be placed in the wells.

The dropped primer solutions are preferably slowly dried by air drying, vacuum drying, freeze drying, or the like. When the substance placed in the wells is an enzyme, a dropped enzyme solution is preferably dried by critical-point drying in order to prevent a decrease or deactivation of activity of the enzyme.

(2-3) Covering with Thin Film

Symbol S₃ shows a step of covering the substance placed in the wells with a thin film 6.

The thin film 6 can be formed by dropping a solution of a material of the thin film 6 on each of the primers placed in the wells, and drying the solution. In order to prevent dissolution or denaturation of the primer, the solution of the material of the thin film 6 is preferably hardened as soon as it contacts the primer. Alternatively, the thin film 6 is preferably formed by placing, in a vacuum evaporation chamber, the substrate layer a₁ having wells containing the primers therein, and evaporating the material of the thin film 6. In performing the evaporation of the thin film 6, appropriate conditions are determined in accordance with the material of the thin film 6, and the thin film 6 is deposited with a general-purpose vacuum evaporation apparatus. When an enzyme is placed in the wells, an apparatus having a mechanism configured to control the temperature of a substrate to be 30° C. or lower is preferably used in order to prevent deactivation of the enzyme.

(2-4) Activation and Bonding of Surfaces of Substrate Layers a₁ and a₂

Symbol S₄ shows a step of activating the surfaces of the substrate layers a₁ and a₂. Symbol S₅ shows a step of bonding between the substrate layers a₁ and a₂.

The bonding between the substrate layers a₁ and a₂ can be performed by, for example, activating the surfaces of the substrate layers by an oxygen plasma treatment or a vacuum ultraviolet light treatment, and then bonding the surfaces to each other. A plastic such as polydimethylsiloxane has a high affinity with glass. When the surfaces of these materials are subjected to an activation treatment and brought into contact with each other, dangling bonds react to each other to form Si—O—Si silanol bonds, which are strong covalent bonds. Thus, joining having a sufficient strength can be achieved. In performing the oxygen plasma treatment or the vacuum ultraviolet light treatment, appropriate conditions are determined in accordance with the materials of the substrate layers.

In this case, when the surfaces of the substrate layers are activated by the oxygen plasma treatment or the vacuum ultraviolet light treatment, the thin film 6 protects the primers, and thus it is possible to prevent degradation and denaturation of the primers caused by the irradiation of plasma or ultraviolet light or the like. In particular, when the substance placed in advance in the wells is an enzyme, the presence of the thin film 6 can effectively prevent a decrease or deactivation of activity of the enzyme caused by the irradiation of plasma or ultraviolet light or the like.

3. Method for Producing Microchip for Nucleic Acid Amplification Reaction According to Modification of this Embodiment

Next, a microchip for a nucleic acid amplification reaction according to a modification of the embodiment described above, and a method for producing the microchip will be described. In this modification, components having substantially the same function and structure as those of the above embodiment are assigned the same reference numerals and symbols, and an overlapping description is omitted.

[Microchip for Nucleic Acid Amplification Reaction]

A microchip A2 (not shown) according to this modification includes an inlet 1, a main channel 2, branch channels 3, wells 41, 42, and 43, an outlet 5, and a thin film 6, all of which have the same function and structure as those of the microchip A according to the above-described embodiment. The microchip A2 is substantially the same as the microchip A according to the above-described embodiment except that at least one of substances necessary for a reaction is fixed on a thin film covering at least one of the remaining substances necessary for the reaction. Here, a description will be made of only the feature that, in a well, at least one of substances necessary for a reaction is fixed on a thin film covering at least one of the remaining substances necessary for the reaction.

Among the substances present in the well, there are no particular restrictions upon which substance is covered with the thin film 6 and which substance is fixed on the thin film 6. A case where a primer is fixed on a thin film 6 covering an enzyme will now be described as an example.

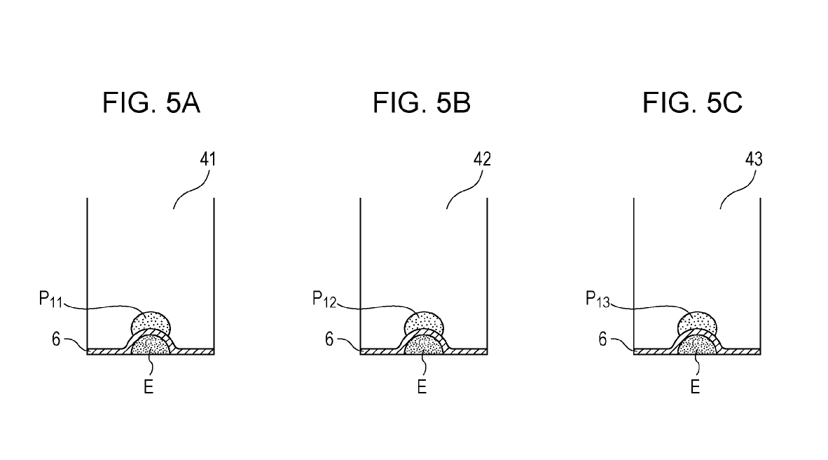

FIGS. 5A to 5C show substances placed in respective wells. FIG. 5A shows a substance placed in the well 41, FIG. 5B shows a substance placed in the well 42, and FIG. 5C shows a substance placed in the well 43. These figures show a case where an enzyme E is placed in the wells 41, 42, and 43, and primers P₁₁, P₁₂, and P₁₃ are each fixed on a thin film 6 covering the enzyme E.

At temperatures lower than the melting temperature, the enzyme E covered with the thin film 6 is present inside the solid thin film 6 so as to be isolated from the outside. At temperatures equal to or higher than the melting temperature, the enzyme E is released from the thin film 6 that is fluidized and collapsed, and can be in contact with the outside.

Each of the primers P₁₁, P₁₂, and P₁₃ is fixed on the corresponding thin film 6, and is redissolved by supplying a sample solution in the wells. As with the primers P₁, P₂, and P₃ described above, the primers P₁₁, P₁₂, and P₁₃ may be primers having the same base sequence. Alternatively, when a plurality of target nucleic acid strands are amplified in the microchip A2, primers having different base sequences are used as the primers P₁₁, P₁₂, and P₁₃.

[Isothermal Nucleic-Acid Amplification Method]

An isothermal nucleic-acid amplification method using the microchip A2 according to this modification is substantially the same as the isothermal nucleic-acid amplification method according to the above embodiment except that at least one substances necessary for a reaction is fixed on a thin film covering at least one of the remaining substances necessary for the reaction.

Specifically, in the microchip A2 according to this modification, first, an enzyme is covered with a thin film 6, and a primer is fixed on the thin film 6. Next, a sample solution at room temperature containing substances (such as dNTP and a buffer solute) other than the enzyme and the primer placed in each well, the substances being necessary for a reaction, is supplied from the inlet 1 to the well. After the sample solution is supplied to the well, the isothermal nucleic-acid amplification method is performed as in the isothermal nucleic-acid amplification method according to the above embodiment.

More specifically, first, a sample solution containing a target nucleic acid strand and substances (such as dNTP and a buffer solute) necessary for a reaction, the substances being other than the primer and the enzyme which are placed in wells in advance and then covered with the thin film 6, is supplied from the inlet 1 to each of the wells. As a result, the primer is redissolved by the sample solution. At this time, the sample solution is maintained at room temperature, and thus the enzyme covered with the thin film 6 is isolated from the sample solution. Accordingly, a reaction caused by mixing of the enzyme and the sample solution does not proceed.

After the sample solution is supplied to each of the wells, the temperature of the sample solution is increased to a reaction temperature. As a result, the thin film 6 is fluidized and collapsed so that the enzyme is released and mixed with the sample solution. Thus, a reaction is started.

As described above, in the microchip A2, the reaction is started by supplying the sample solution to each of the wells and then increasing the temperature of the sample solution to the reaction temperature. Thus, the reaction time can be strictly controlled in the microchip A2.

Furthermore, in the isothermal nucleic-acid amplification method according to this modification, a reaction is conducted after an enzyme is covered with a thin film and a primer is fixed on the thin film. Accordingly, in the case where an enzyme and different types of primers are placed in a plurality of wells, determination of a contagium or the like can be easily conducted while accurately controlling the reaction time.

[Method for Producing Microchip for Nucleic Acid Amplification Reaction]

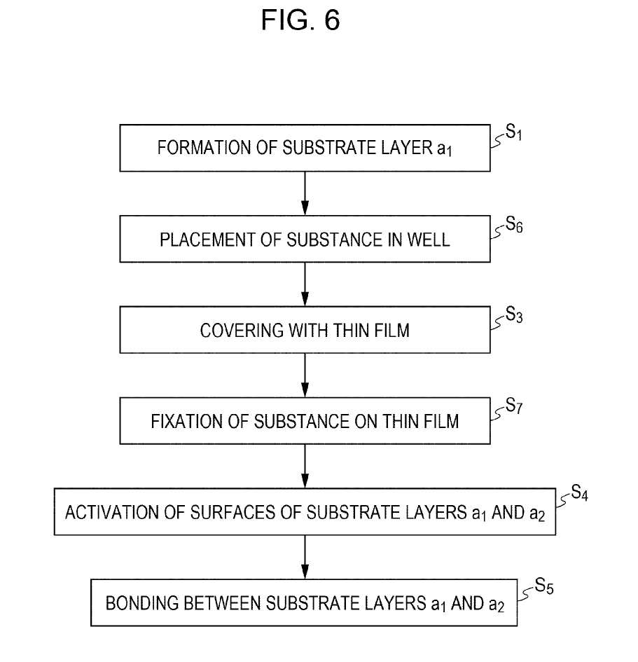

A method for producing the microchip according to this modification will now be described with reference to the flowchart shown in FIG. 6. A description will be made by taking the above-described microchip A2 as an example. However, the steps shown by symbols S₁, S₃, S₄, and S₅ are substantially the same as those in the method for producing the microchip A for a nucleic acid amplification reaction according to the above embodiment. Therefore, only steps shown by symbols S₆ and S₇ will be described here.

In FIG. 6, symbol S₆ shows a step of placing a substance necessary for a reaction in the wells. In this step, a solution of an enzyme E is dropped in the wells 41, 42, and 43 and dried, thus placing the enzyme E in the wells. As described above, the substance placed in the wells is not limited to an enzyme. Alternatively, for example, dNTP, a primer, or a buffer solute may be placed in the wells. As described in this modification, when the substance placed in the wells is an enzyme, the dropped enzyme solution is preferably dried by critical-point drying in order to prevent a decrease or deactivation of activity of the enzyme.

Symbol S₇ shows a step of fixing a substance necessary for the reaction on a thin film 6. In this step, solutions of primers P₁₁, P₁₂, and P₁₃ are dropped on the corresponding thin film 6 and dried, thus placing the primers in the wells. Also in this step, as described above, the substance placed in the wells is not limited to the primers. Alternatively, the substance may be dNTP, an enzyme, or a buffer solute, that is, a substance other than the substance covered with the thin film 6, the substance being necessary for the reaction. The dropped primer solutions are preferably slowly dried by air drying, vacuum drying, freeze drying, or the like.

### EXAMPLES

### Example 1

### 1. Preparation of Microchip for Isothermal Nucleic-Acid Amplification Reaction

(1) Fixation of Primers

For a template nucleic acid strand, six types of primers for LAMP shown in Table 1 were designed. Next, 0.5 μL of a primer solution was dropped in a well provided in a PDMS substrate. The primers were fixed in the well by vacuum drying (room temperature, 0.1 Pa, 5 minutes).

TABLE 1

| | | Sequence |
|---|---|---|
| Primer | Base sequence | No. |
| Forward inner | 5′-TACAC CTTTG TTCGA GTCAT GATGA AAGGT TTGAG ATATT CCCA-3′ | 1 |
| primer(FIP) | | |
| Backward inner | 5′-CTCAT GCTGG AGCAA AAAGC TTCAT TTGCT GAGCT TTGGG T-3′ | 2 |
| primer (BIP) | | |
| F3 | 5′-GCAAT TGAGC TCAGT GTCAT-3′ | 3 |
| B3 | 5′-TCTTT CCCTT TATCA TTAAT GTAGG-3′ | 4 |
| LF | 5′-TGGGC CATGA ACTTG TCT-3′ | 5 |
| LB | 5′-GGCTA GTTAA AAAAG GAAAT TCA-3′ | 6 |

(2) Covering with Thin Film

Stearic acid was placed on an evaporation boat made of tungsten, and a thermoelectric heater was connected to the boat. The boat was placed in a vacuum evaporation chamber. The substrate having the well in which the primers had been fixed was placed in the vacuum evaporation chamber. The chamber was evacuated to a pressure of 1.0×10-6 Ton, and the tungsten boat was heated by supplying a current to the thermoelectric heater. A shutter was opened at the time when the stearic acid became a solution on the evaporation boat to start evaporation.

The evaporation was conducted while heating the stearic acid (melting point: 69° C.) at 85° C. so that the vacuum saturated vapor pressure was 0.1 Torr. The evaporation was conducted at a substrate temperature of 30° C. so that the film thickness was 100 nm.

After the evaporation, the substrate was taken out from the vacuum evaporation chamber. Formation of a stearic acid film on the surface of the primers fixed in the well was confirmed by the presence of an interference color. Subsequently, the surface of the substrate was hydrophilized by DP ashing (O2: 10 cc, 100 W, 30 seconds), and was bonded to a cover glass to prepare a microchip.

### 2. LAMP Reaction

A template nucleic acid strand, an enzyme, an intercalator fluorescent dye (SYBR Green I, manufactured by Molecular Probes Inc.), a nucleic acid monomer, a buffer were mixed to prepare a reaction solution. The reaction solution was injected into a channel of the microchip prepared in Example 1 to introduce into the well. The microchip was set in a fluorescence detection device including a fluorescence-detecting portion and a heating portion for each well, and a reaction was started. The amount of amplification of the target nucleic acid strand was measured in real time on the basis of the fluorescence intensity of the fluorescent dye.

An amplified nucleic acid strand was detected after about 10 minutes from the start of the LAMP reaction. Thus, a good quantitativity of the target nucleic acid strand was confirmed.

### Example 2

### Examination of Materials of Thin Film

LAMP Reaction

A solution of the primers shown in Table 1 was dispensed in microtubes, and the primers were fixed in the microtubes by vacuum drying (room temperature, 0.1 Pa, 5 minutes). Stearic acid or a paraffin wax (Paraffin 115, 125, 130, 135, or 140) was dispensed in the microtubes, and a reaction solution was then added thereto.

A solution of an enzyme was dispensed in microtubes, and the enzyme was fixed in the microtubes by vacuum drying (room temperature, 0.1 Pa, 5 minutes). Stearic acid or a paraffin wax (Paraffin 115, 125, 130, 135, or 140) was dispensed in the microtubes, and a primer solution and a reaction solution containing reagents other then the enzyme were added thereto.

The microtubes were set in a fluorescence detection device including a fluorescence-detecting portion and a heating portion for each tube, and a reaction was started. The amount of amplification of a target nucleic acid strand was measured in real time on the basis of the fluorescence intensity of a fluorescent dye.

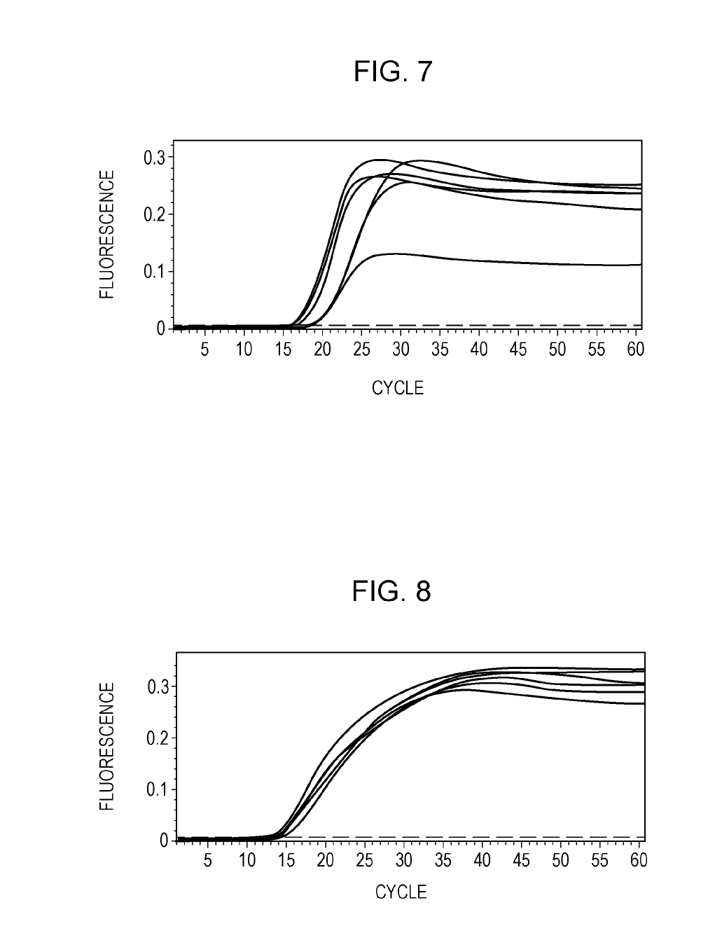

FIGS. 7 and 8 show the results. FIG. 7 shows the results of reaction systems in which the fixed primers were covered with stearic acid or a paraffin wax. FIG. 8 shows the results of reaction systems in which the enzyme was fixed, and the fixed enzyme was covered with stearic acid or a paraffin wax. In all the reaction systems, an amplified nucleic acid strand was detected after about 10 to 15 minutes from the start of the LAMP reaction. Thus, it was confirmed that paraffin waxes also had no reaction inhibiting property.

The present application contains subject matter related to that disclosed in Japanese Priority Patent Application JP 2010-141318 filed in the Japan Patent Office on Jun. 22, 2010 and Japanese Priority Patent Application JP 2010-195780 filed in the Japan Patent Office on Sep. 1, 2010, the entire contents of which are hereby incorporated by reference.

It should be understood by those skilled in the art that various modifications, combinations, sub-combinations and alterations may occur depending on design requirements and other factors insofar as they are within the scope of the appended claims or the equivalents thereof.

### SEQUENCE LISTING

201009011604502030_A163_{—}305969JP00_{—}12010195780_AAA_{—}1.app

It should be understood that various changes and modifications to the presently preferred embodiments described herein will be apparent to those skilled in the art. Such changes and modifications can be made without departing from the spirit and scope and without diminishing its intended advantages. It is therefore intended that such changes and modifications be covered by the appended claims.

The application is claimed as follows:

1. A microchip for an isothermal nucleic-acid amplification reaction wherein at least one of substances necessary for an isothermal amplification reaction of a nucleic acid is present in a reaction region functioning as a reaction field of the reaction, the at least one of the substances being covered with a thin film that melts at a temperature higher than room temperature and lower than a reaction temperature of the reaction.

2. The microchip according to claim 1, wherein at least one of the remaining substances necessary for the reaction is fixed on the thin film covering the at least one of the substances necessary for the reaction.

3. The microchip according to claim 1, wherein the thin film is formed by evaporation of stearic acid or a paraffin wax.

4. The microchip according to claim 3, wherein the at least one of the substances present in the reaction region is at least one selected from an oligonucleotide primer, an enzyme, and a nucleic acid monomer.

5. The microchip according to claim 2, wherein an oligonucleotide primer is fixed on the thin film covering an enzyme.

6. The microchip according to claim 1, wherein the microchip includes
an inlet into which a liquid is introduced from the outside,
a plurality of the reaction regions, and
a channel configured to supply the liquid introduced from the inlet to the reaction regions.

7. A method for producing a microchip for an isothermal nucleic-acid amplification reaction, comprising:
covering at least one of substances necessary for an isothermal amplification reaction of a nucleic acid, the at least one of the substances being placed in a reaction region functioning as a reaction field of the reaction, with a thin film that melts at a temperature higher than room temperature and lower than a reaction temperature of the reaction.

8. An isothermal nucleic-acid amplification method comprising:
into a reaction region where at least one of substances necessary for an isothermal amplification reaction of a nucleic acid is present, the at least one of the substances being covered with a thin film that melts at a temperature higher than room temperature and lower than a reaction temperature of the reaction, introducing the remaining substances necessary for the reaction; and
increasing the temperature to the reaction temperature.
